(19) United States
(12) Patent Application Publication
(10) Pub. No.: US 2025/0269115 A1
(43) Pub. Date: Aug. 28, 2025

(54) CLOSURE PIECE FOR A MEDICAL HOLLOW BODY, MEDICAL HOLLOW BODY HAVING SUCH A CLOSURE PIECE, AND METHOD FOR PRODUCING SUCH A CLOSURE PIECE

(71) Applicant: VETTER PHARMA-FERTIGUNG GMBH & CO. KG, Ravensburg (DE)

(72) Inventor: Petra HUND, Ravensburg / Berg (DE)

(21) Appl. No.: 18/702,988

(22) PCT Filed: Oct. 20, 2022
(86) PCT No.: PCT/EP2022/079241

§ 371 Date: Apr. 19, 2024

(30) Foreign Application Priority Data
Oct. 21, 2021 (DE) 102021127435.4

(51) Int. Cl. A61M 5/31

(52) U.S. Cl. CPC A61M 5/31; A61M 2005/3104; A61M 2005/3106; A61M 2205/0216; A61M 2207/00

(57) ABSTRACT

The invention relates to a cap for a medical hollow body, with
an engaging element which is configured such that it engages at least partially in a distal opening of the hollow medical body when the cap is arranged on the hollow medical body in the closing position, wherein
the engaging element has at least one first groove, wherein a groove direction associated with the at least one first groove is aligned along an axial direction of the medical hollow body when the cap is arranged on the medical hollow body in the closing position.

### CROSS REFERENCE TO RELATED APPLICATIONS

The present application is a national stage application of International Application No. PCT/EP2022/079241, filed Oct. 20, 2022, which claims priority to German Patent Application No. 102021127435.4, filed Oct. 21, 2021, the entire contents of which are incorporated herein by reference in their entirety.

### DESCRIPTION

The invention relates to a cap for a medical hollow body, a medical hollow body with such a cap and a method for producing such a cap.

It is known that in hollow medical bodies prefilled with a product, in particular in prefilled disposable syringes, a pressure difference can form between an interior of the hollow medical body and an exterior of the hollow medical body, in particular an overpressure in the interior of the hollow medical body. It is possible that this pressure difference, in particular this overpressure, is formed due to a manufacturing process, in particular during an arrangement of a stopper and/or a piston rod, and/or a change in the air pressure during transport and/or storage of the prefilled medical hollow body.

Known caps for the pre-filled medical hollow bodies, in particular, have at least one sealing element which is configured such that it covers a distal opening of the medical hollow body in a sealing manner when the cap is arranged on the medical hollow body in a closing position.

When the medical hollow body is opened, in particular when the cap is removed, drop formation of the product at the distal opening of the medical hollow body can occur due to the excess pressure in the medical hollow body, in particular the prefilled medical hollow body, and the associated immediate equalisation of pressure.

The disadvantage of this is that the drops swell out of the medical hollow body when it is opened, thus reducing the amount of product available for administration to a patient. Furthermore, it is possible that the droplet wets a thread and/or a cone of the hollow medical body, which adversely affects, in particular reduces, the adhesion between the thread or the cone and a device for applying the product of the hollow medical body.

The invention is therefore based on the task of creating a cap for a hollow medical body, a hollow medical body with such a cap and a method for producing such a cap, wherein the aforementioned disadvantages are at least partially eliminated, preferably avoided.

The task is solved by providing the present technical teaching, in particular the teaching of the independent claims and the embodiments disclosed in the dependent claims and the description.

In particular, the task is solved by providing a cap for a medical hollow body, wherein the cap comprises an engaging element. The engaging element is configured such that it engages at least partially—in particular with a particularly conically shaped engaging protrusion—in a distal opening of the hollow medical body when the cap is arranged on the hollow medical body in the closing position. The engaging element has at least one first groove, wherein a groove direction associated with the at least one first groove is aligned along an axial direction of the medical hollow body when the cap is arranged on the medical hollow body in the closing position. In particular, the first groove is configured on the engaging protrusion.

In the context of the present technical teaching, the groove direction associated with a groove is a direction of a straight line extending from a starting point of the groove to an end point of the groove, wherein the starting point of the groove and the end point of the groove are preferably spaced apart along the longest extent of the groove.

The groove direction is aligned along the axial direction of the hollow medical body if the groove direction and the axial direction of the hollow medical body enclose an angle of less than 90°, preferably an angle of less than or equal to 45°.

Advantageously, the cap, in particular the engaging element, closes the hollow medical body in a sealing manner when the cap is arranged on the hollow medical body in the closing position. Advantageously, the engaging element in particular is in contact with a product present in the hollow medical body, in particular in fluidic contact, when the cap is arranged on the hollow medical body in the closing position.

Advantageously, the groove forms a fluidic connection between an interior of the medical hollow body and an exterior of the medical hollow body when the cap is released from the closing position and preferably the engaging element still engages in the distal opening of the medical hollow body. By means of this fluidic connection, a slower pressure equalisation is advantageously possible compared to the known caps. This can advantageously at least reduce the formation of drops at the distal opening of the hollow medical body, which can reduce a reduction in the product present in the hollow medical body.

Preferably, the engaging element is configured in such a way that it rests sealingly against or on an end face of the distal area of the hollow medical body when the cap is arranged on the hollow medical body in its closing position. The end face of the distal area is in particular an end face of the distal opening.

Preferably, the part of the engaging element that engages in the distal opening of the hollow medical body—in particular the engaging protrusion, which is configured conically in particular—lies in a sealing manner against the hollow medical body, in particular against an inner wall of the hollow medical body, at least in partial areas, in particular outside the first groove.

The axial direction of the hollow medical body preferably extends along a direction of longest extension of the hollow medical body, preferably along an axis of symmetry of the preferably rotationally symmetrical hollow medical body. A radial direction is perpendicular to the axial direction. A circumferential direction concentrically embraces the axial direction.

In particular, the engaging element has an underside, wherein the underside faces the end face of the distal opening of the hollow medical body at least partially when the cap is arranged on the hollow medical body in the closing position.

Preferably, the hollow medical body is configured as a syringe.

Preferably, the product present in the hollow medical body is a medical or pharmaceutical product, in particular a medical or pharmaceutical liquid.

According to a further development of the invention, it is provided that the at least one first groove extends straight and has a groove longitudinal extension as the groove direction. Preferably, when the cap is arranged on the medical hollow body in the closing position, the groove longitudinal extension and the axial direction of the medical hollow body form an angle of 0° to 25°.

Preferably, the groove longitudinal extension and the axial direction of the hollow medical body are aligned parallel to each other when the cap is arranged on the hollow medical body in the closing position.

According to a further development of the invention, it is provided that the at least one first groove is configured along a spatial curve, in particular a helical line or a conical spiral. The spatial curve extends along a winding axis. Preferably, the winding axis of the at least one first groove and the axial direction of the hollow medical body are parallel to each other when the cap is arranged on the hollow medical body in the closing position.

The groove direction of the at least one first groove is determined by means of a starting point of the spatial curve and an end point of the spatial curve. In particular, the groove direction of the at least one first groove, which extends along the spatial curve, and the axial direction of the hollow medical body include an angle of less than 90°, particularly preferably of less than or equal to 45°, when the cap is arranged on the hollow medical body in the closing position.

Preferably, the first groove configured as a conical spiral has a pitch height of 0.5 mm to 2 mm, preferably 1 mm. Alternatively or additionally, the spiral-shaped first groove has at least one turn, preferably from 1 turn to 3 turns, particularly preferably 1.75 turns. Alternatively or additionally, the spiral-shaped first groove comprises a taper of 0° to 45°, preferably 20°.

Alternatively or additionally, a groove width of the first groove has a taper of 0° to 45°, preferably 20°.

According to a further development of the invention, it is provided that the engaging element—in particular the engaging protrusion, which is configured conically in particular—has at least one second groove which extends in a plane, wherein a normal direction of the plane when the cap is arranged on the hollow medical body in the closing position and the axial direction of the hollow medical body enclose an angle of 0° to 18°.

Preferably, the normal direction of the plane and the groove longitudinal extension of the first groove are aligned parallel to each other. Alternatively or additionally, the normal direction of the plane when the cap is arranged on the hollow medical body in the closing position and the axial direction of the hollow medical body are preferably aligned parallel to each other.

While the cap is released from the closing position and preferably the engaging element still engages in the distal opening of the hollow medical body, gas is advantageously guided from the interior of the hollow medical body to the second groove by means of the first groove, irrespective of the way in which—in particular in which direction or at which angle in relation to the axial direction of the hollow medical body—the cap is released from the closing position, wherein in particular the second groove comes into flow connection with the exterior of the hollow medical body. Thus, regardless of the way in which the cap is removed from the hollow medical body, a slower pressure equalisation is possible compared to the known caps.

According to a further development of the invention, it is provided that the cap comprises a sealing element. The sealing element is configured in such a way that it is in sealing contact with at least a distal area of the hollow medical body when the cap is arranged on the hollow medical body in a closing position.

Advantageously, the sealing element is not in contact with the product present in the hollow medical body when the cap is arranged on the hollow medical body in the closing position, in particular when the sealing element and the engaging element are configured in two-pieces.

In a preferred configuration, the sealing element is configured such that it sealingly surrounds at least the distal area of the hollow medical body when the cap is arranged on the hollow medical body in the closing position.

According to a further embodiment of the invention, it is provided that the sealing element and the engaging element are configured as a single-piece, preferably as material uniform. Advantageously, this makes it possible to manufacture the sealing element and the engaging element in one process step, saving time and costs.

According to a further development of the invention, it is provided that the sealing element and the engaging element are configured in two-pieces. Advantageously, this makes it possible to manufacture the sealing element and the engaging element from different materials in a simple manner and, in particular, to adapt them to special requirements. Advantageously, it is also possible to manufacture the engaging element from a material that cannot be processed using an injection moulding method.

In one embodiment of the cap, the engaging element is accommodated at least partially in the sealing element, in particular in a notch in the sealing element provided for this purpose. In particular, the sealing element surrounds the engaging element in the circumferential direction.

In particular, it is advantageous to select the material of the engaging element due to a particularly permanent contact with the product present in the medical hollow body, in particular during transport and/or storage, in such a way that the material is stable, in particular chemically stable, with respect to the product and preferably does not migrate into the product.

According to a further development of the invention, it is provided that the engaging element has a sealing lip extending in the circumferential direction, which is arranged on the engaging element in such a way that the sealing lip comes to rest on the end face of the distal opening of the hollow medical body when the cap is arranged on the hollow medical body in the closing position.

Advantageously, the sealing lip thus forms a further seal, in particular in addition to the area of the engaging element engaging in the distal opening of the hollow medical body, between the interior of the hollow medical body, in particular the product, and the exterior of the hollow medical body.

In particular, the sealing lip is arranged on the underside of the engaging element. Thus, the sealing lip faces the end face of the distal opening of the hollow medical body at least partially when the cap is arranged on the hollow medical body in the closing position.

According to a further embodiment of the invention, it is provided that the cap comprises a base body. The engaging element is arranged at least partially within the base body. Alternatively or additionally, the sealing element is arranged at least partially within the base body.

Advantageously, the base body is not in contact with the product present in the hollow medical body when the cap is arranged on the hollow medical body in the closing position.

Preferably, the base body embraces the sealing element and the engaging element.

Particularly preferably, the sealing element is arranged in the base body in such a way that it is in sealing contact with at least the distal area of the hollow medical body when the cap is arranged on the hollow medical body in a closing position. Alternatively or additionally, the engaging element is particularly preferably arranged in the base body in such a way that it engages at least partially—in particular with the particularly conically configured engaging protrusion—in a distal opening of the hollow medical body when the cap is arranged on the hollow medical body in the closing position.

According to a further development of the invention, it is provided that the base body has an internal thread. The internal thread engages around a distal area of the hollow medical body when the cap is arranged on the hollow medical body in the closing position. The thread is preferably a Luer thread.

Advantageously, it is thus possible in a simple manner to connect a device for applying the product, in particular an adapter or a cannula, to the hollow medical body by means of the cap.

According to a further development of the invention, it is provided that the sealing element and the engaging element are arranged in the base body in such a way that they are spaced apart from one another in at least one first area along the groove longitudinal extension, wherein they come to rest on one another in at least one second area.

Advantageously, a position of the engaging element relative to the sealing element and the base body is fixed by means of the at least one second area. In addition, when the cap is arranged on the hollow medical body in the closing position, the second area advantageously serves to hold down the engaging element and preferably to press it onto the distal opening of the hollow medical body.

Preferably, the second area is designed such that manufacturing tolerances of at least one element selected from a group consisting of the base body, the sealing element and the engaging element are compensated.

In particular, the at least one first area is a notch, in particular a cavity, between the engaging element and the sealing element.

Advantageously, the at least one first area thus enables that, when the cap is arranged on the medical hollow body in the closing position, the engaging element can avoid an existing pressure during a press fit with the medical hollow body by the engaging element being at least partially displaced into the at least one first area, and the engaging element is thus at least only partially compressed.

Advantageously, when the sealing lip is arranged on the hollow medical body in the closing position, it is possible to realise the tightness of the hollow medical body even when the contact pressure of the engaging element on the end face of the distal opening of the hollow medical body is reduced. In particular, environmental influences and/or during a sterilisation process can lead to a reduction in the contact pressure.

According to a further development of the invention, it is provided that the base body comprises an upper part and a lower part, wherein preferably the lower part comprises the internal thread. The upper part and the lower part are connected to each other by means of a plurality of ribs.

Advantageously, the plurality of ribs makes it easy to separate the upper part and the lower part from each other, in particular irreversibly. In particular, it is advantageously possible to reduce the force required to open the cap and at the same time provide tamper detection.

Preferably, the internal thread, in particular the Luer thread, and the base body, in particular the lower part, are configured in two-pieces. Alternatively, the internal thread, in particular the Luer thread, and the base body, in particular the lower part, are preferably configured as a single-piece.

In a preferred configuration, the upper part and the lower part are configured such that, while the cap is released from the closing position, the upper part and the lower part are separated from each other, in particular irreversibly, wherein the lower part remains arranged on the medical hollow body. In addition, the upper part, the sealing element and the engaging element are connected to each other in such a way that, while the cap is released from the closing position, the sealing element and the engaging element are also separated from the medical hollow body together with the upper part.

Preferably, an axial direction of the upper part, an axial direction of the lower part and the axial direction of the medical hollow body are aligned parallel to each other in a closing position, in particular identical. Particularly preferably, the cap is released from the closing position by tilting the upper part relative to the axial direction of the hollow medical body and thus in particular relative to the axial direction of the lower part in an opening direction orthogonal to the axial direction of the lower part, so that at least one rib of the plurality of ribs is stretched and at least one rib of the plurality of ribs is crushed. In particular, all ribs of the plurality of ribs are irreversibly destroyed during tilting, so that the upper part and the lower part can be separated from each other. Particularly preferably, the opening direction is orientated opposite a groove opening direction of the first groove.

In the context of the present technical teaching, the groove opening direction is orientated orthogonally to a plane in which the groove is inserted. In addition thereto, the groove opening direction is aligned orthogonally to the groove longitudinal extension.

Preferably, the upper part and the lower part of the base body are connected by means of a maximum of eight ribs.

In a preferred configuration, the base body comprises the upper part, the lower part and the plurality of ribs connecting the upper part and the lower part. In addition, the engaging element comprises the sealing lip. When the cap is arranged on the hollow medical body in the closing position, the sealing lip makes it possible to reduce the contact pressure of the engaging element on the end face of the distal opening of the hollow medical body. This can advantageously reduce tension on the ribs, which can advantageously reduce deformation of the ribs during heating—in particular during sterilisation of the hollow medical body sealed with the cap.

According to a further development of the invention, it is provided that the cap has a retaining element which is arranged on the base body in such a way that it preferably grips the hollow medical body in a form and/or force-fit manner when the cap is arranged on the hollow medical body in the closing position. In addition, the retaining element has at least one anti-rotation section which engages in a notch in the base body, in particular in the lower part of the base body.

Advantageously, the retaining element enables the lower part to remain arranged on the medical hollow body while the cap is released from the closing position. In addition, the anti-rotation section fixes the base body of the cap to the retaining element so that it cannot rotate.

Preferably, the retaining element is arranged on the base body before the cap is arranged in the closing position on the medical hollow body.

Particularly preferably, the retaining element is latched to the base body before the cap is arranged in the closing position on the medical hollow body.

In a preferred configuration, the retaining element and the plurality of ribs advantageously interact in such a way that, while the cap is released from the closing position, the upper part and the lower part are separated from each other, in particular irreversibly, and the lower part remains arranged on the hollow medical body.

According to a further development of the invention, it is provided that the base body and the sealing element are produced by means of a two-component injection moulding process. Alternatively, the base body, the sealing element and the engaging element are produced by means of a multi-component injection moulding process, in particular the two-component injection moulding process.

Advantageously, it is thus possible to manufacture the base body, the sealing element and preferably the engaging element in one process step, saving time and money. Advantageously, an additional process step is not required to connect the base body, the sealing element and preferably the engaging element to one another.

According to a further development of the invention, it is provided that the sealing element comprises at least one thermoplastic elastomer (TPE). Alternatively, it is provided that the sealing element consists of at least one thermoplastic elastomer (TPE).

Advantageously, a thermoplastic elastomer can be processed in a simple manner by means of an injection moulding method.

According to a further development of the invention, it is provided that the engaging element comprises at least one elastomer. Alternatively, it is provided that the engaging element consists of at least one elastomer. Advantageously, elastomers are stable, in particular chemically stable, with respect to a plurality of products that may be present inside the medical hollow body. In addition, an engaging element made of at least one elastomer can advantageously be used to realise a good seal between the inside and the outside of the medical hollow body.

In particular, the engaging element preferably comprises at least one rubber material. Alternatively, the engaging element preferably consists of at least one rubber material.

Preferably, the at least one elastomer is a vulcanisate of a rubber. Alternatively, the at least one elastomer is particularly preferably a vulcanisate of a silicone rubber. Alternatively or additionally, the at least one elastomer is preferably selected from a group consisting of a halogenated butyl rubber, in particular a bromobutyl rubber, a chlorobutyl rubber, a polyisoprene rubber, a polybutadiene rubber, an ethylene-propylene copolymer rubber, an ethylene-propylene-diene copolymer rubber, a chlorosulphonated polyethylene rubber, an ethylene-vinyl acetate copolymer rubber, a styrene-isoprene copolymer rubber, a fluoroelastomer rubber, a perfluoroelastomer rubber, a tetrafluoroethylene rubber, a propylene rubber, a butyl rubber, an isoprene rubber, a butadiene rubber, an ethylene-propylene terpolymer rubber, a silicone rubber and a styrene-butadiene copolymer rubber.

Advantageously, the engaging element comprising at least one elastomer, in particular at least one rubber material, or consisting of at least one elastomer, in particular at least one rubber material, prevents the contents of the medical hollow body from coming into primary contact with the sealing element and thus possibly with a less suitable material. In particular, the engaging element, which consists of a material different from the material of the sealing element, ensures that the contents of the medical hollow body do not come into primary contact with a thermoplastic elastomer.

In a preferred configuration, the engaging element and the sealing element are configured as a single-piece and of the same material. Preferably, the engaging element and the sealing element comprise at least one thermoplastic elastomer (TPE). Alternatively, it is preferable that the engaging element and the sealing element consist of at least one thermoplastic elastomer (TPE). Alternatively, it is preferable that the engaging element and the sealing element comprise at least one elastomer, in particular at least one rubber material. Alternatively, it is preferably provided that the engaging element and the sealing element comprise at least one elastomer, in particular at least one rubber material.

In a further preferred configuration, the engaging element and the sealing element are configured as a single-piece. In particular, the sealing element is preferably moulded onto the engaging element. Alternatively or additionally, the engaging element and the sealing element are in particular fused together. Preferably, the sealing element comprises at least one thermoplastic elastomer (TPE). Alternatively, it is preferably provided that the sealing element consists of at least one thermoplastic elastomer (TPE). Alternatively or additionally, the engaging element preferably comprises at least one elastomer, in particular at least one rubber material. Alternatively or additionally, it is preferably provided that the engaging element consists of at least one elastomer, in particular at least one rubber material.

According to a further development of the invention, it is provided that the base body comprises a first gripping mould and a second gripping mould, wherein the first gripping mould and the second gripping mould are configured on a lateral surface of the base body. Preferably, the first gripping mould and the second gripping mould are arranged diametrically opposite one another. Alternatively or additionally, the first gripping mould and the second gripping mould are preferably configured on the lower part of the base body.

Advantageously, it is possible by means of the gripping moulds to grip the cap in a simple manner and in particular to hold the lower part in order to arrange the cap on a medical hollow body in the closing position or to separate the lower part and the upper part of the base body, in particular irreversibly, when the cap is opened.

Advantageously, the gripping moulds additionally improve the visual and haptic impression and ergonomics of the cap.

According to a further development of the invention, it is provided that the base body comprises at least one recess. In addition, the sealing element comprises at least one anchoring element, wherein the sealing element is arranged in the base body in such a way that the at least one anchoring element engages in the at least one recess.

Advantageously, it is possible to connect the base body and the sealing element to one another in a simple manner by means of the at least one anchoring element in operative connection with the at least one recess.

Preferably, the at least one anchoring element is configured such that it protrudes from the at least one recess in an axial direction—in particular in the axial direction of the base body. Alternatively or additionally, the at least one anchoring element is preferably configured such that it protrudes from the at least one recess in a radial direction—in particular in a radial direction of the base body.

In addition, the at least one recess is particularly preferably configured on the upper part of the base body.

Advantageously, it is possible by means of the at least one anchoring element, which protrudes from the at least one recess, to grip the cap in a simple manner and in particular to hold the upper part in order to arrange the cap on a medical hollow body in the closing position or to separate the lower part and the upper part of the base body, in particular irreversibly, when the cap is opened.

Preferably, the at least one recess and the at least one anchoring element are configured in the shape of an arrow, so that the preferred opening direction of the cap can be represented by means of the at least one recess and the at least one anchoring element.

The task is also solved by creating a medical hollow body with a cap according to the invention or a cap according to one or more of the embodiments described above. In particular, the advantages already explained in connection with the cap arise in connection with the medical hollow body.

The task is also solved by creating a method for producing a cap according to the invention or a cap according to one or more of the embodiments described above, wherein the base body and the sealing element are produced by means of a two-component injection moulding process. Alternatively, the base body, the sealing element and the engaging element are preferably produced by means of the two-component injection moulding process or the multi-component injection moulding process. As part of the method, in particular the cap according to the invention or a cap according to one or more of the embodiments described above is obtained. In connection with the method, the advantages already explained in connection with the cap and the medical hollow body arise in particular.

The invention is explained in more detail below with reference to the drawing. It shows:

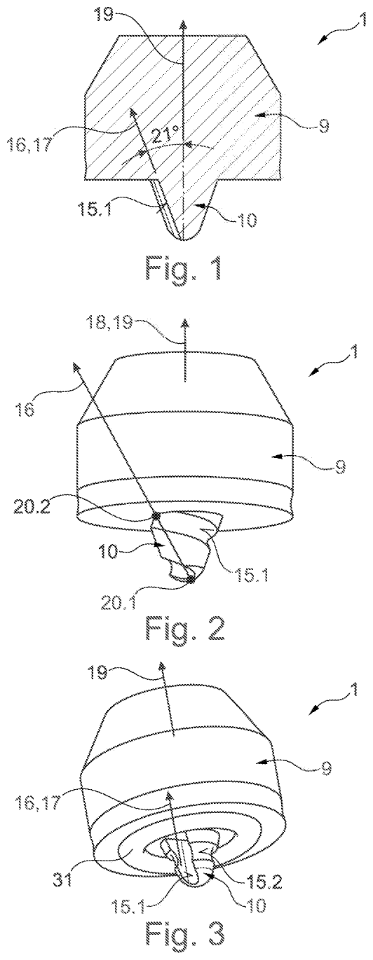

FIG. 1 a schematic representation of a first embodiment of a cap,

FIG. 2 a schematic representation of a second embodiment of the cap,

FIG. 3 a schematic representation of a third embodiment of the cap,

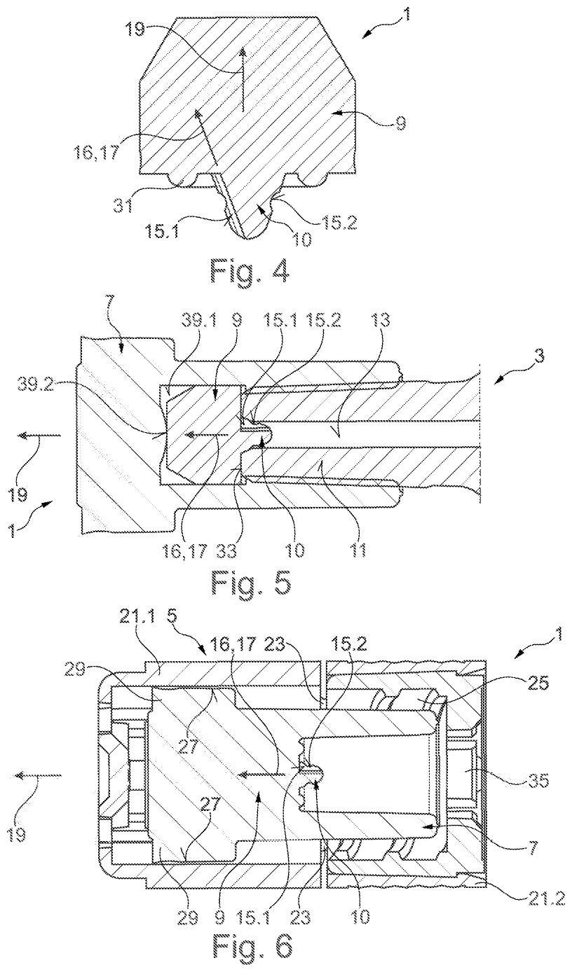

FIG. 4 a schematic representation of a fourth embodiment of the cap,

FIG. 5 a schematic representation of a first embodiment of a medical hollow body,

FIG. 6 a schematic representation of a fifth embodiment of the cap,

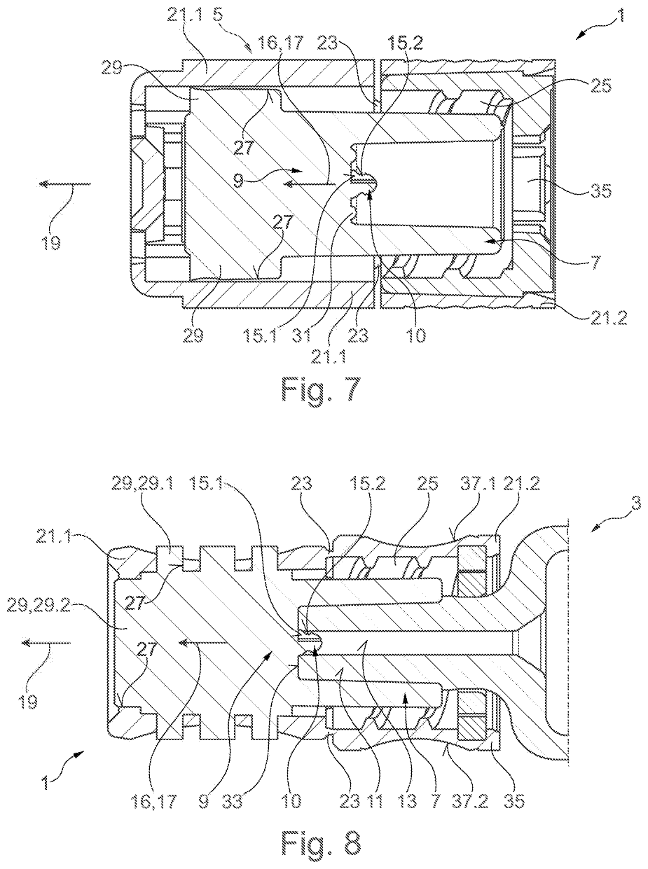

FIG. 7 a schematic representation of a sixth embodiment of the cap,

FIG. 8 a schematic representation of a second embodiment of the medical hollow body,

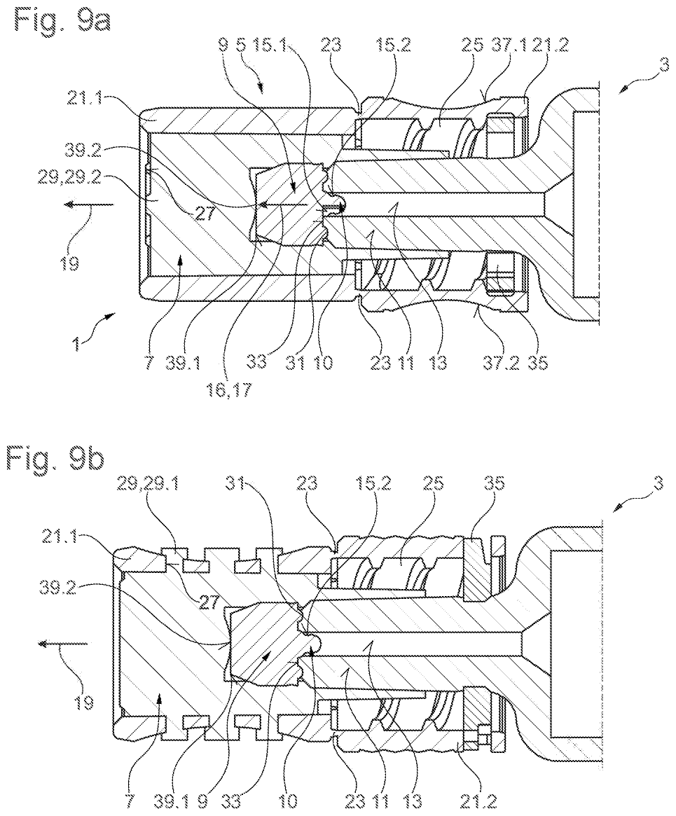

FIGS. 9a and 9b a schematic representation of a third embodiment of the medical hollow body,

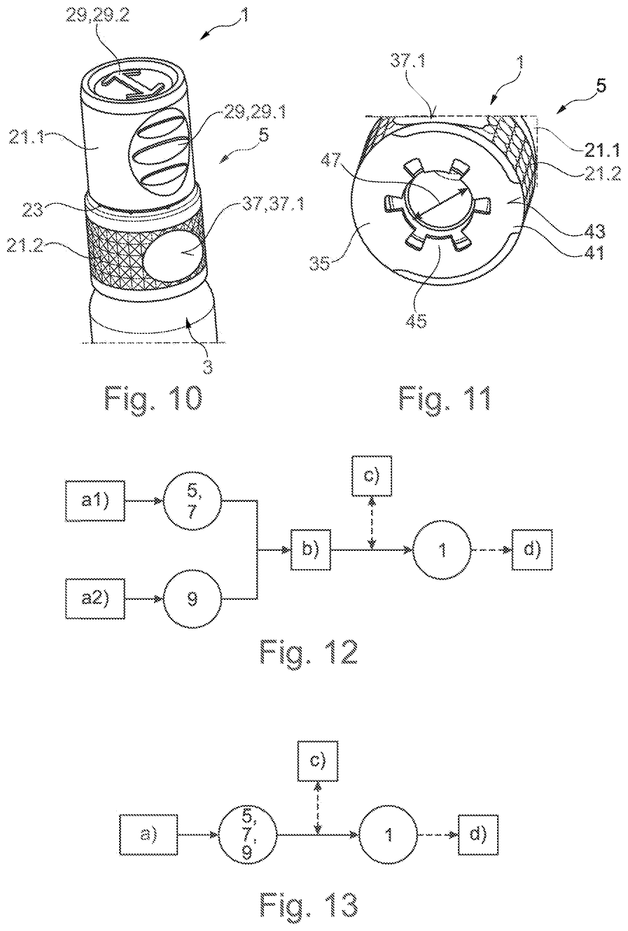

FIG. 10 a schematic representation of a fourth embodiment of the medical hollow body,

FIG. 11 a schematic sectional view of a seventh embodiment of the cap,

FIG. 12 a flow diagram of a first embodiment of a method for manufacturing the cap, and

FIG. 13 a flow diagram of a second embodiment of the method for manufacturing the cap.

FIG. 1 shows a schematic representation of a first embodiment of a cap 1.

The cap 1 has an engaging element 9 which is configured such that it engages at least partially—in particular with an engaging protrusion 10—in a distal opening 13 of a hollow medical body 3 when the cap 1 is arranged on the hollow medical body 3 in the closing position. The engaging element 9 has—in particular on the engaging protrusion 10—at least one first groove 15.1 with a groove direction 16 assigned to the at least one first groove 15.1. When the cap 1 is arranged on the medical hollow body 3 in the closing position, the groove direction 16 is aligned along an axial direction 19 of the medical hollow body 3.

Preferably, the at least one first groove 15.1 extends straight and has a groove longitudinal extension 17 as the groove direction 16.

Particularly preferably, the groove longitudinal extension 17 and the axial direction 19 of the hollow medical body 3 form an angle of 0° to 25°, in particular 21°, when the cap 1 is arranged on the hollow medical body 3 in the closing position.

FIG. 2 shows a schematic representation of a second embodiment of the cap 1 with the engaging element 9.

Identical and functionally identical elements are provided with the same reference signs in all figures, so that reference is made to the previous description in each case.

The at least one first groove 15.1 is configured along a conical spiral, in particular the first groove 15.1 is configured as a conical spiral. Preferably, a winding axis 18 of the at least one first groove 15.1 and the axial direction 19 of the medical hollow body 3 are parallel to each other when the cap 1 is arranged on the medical hollow body 3 in the closing position.

The groove direction 16 associated with the at least one first groove 15.1 is a direction of a straight line extending from a starting point 20.1 of the at least one first groove 15.1 to an end point 20.2 of the at least one first groove 15.1, wherein the starting point 20.1 and the end point 20.2 are spaced apart along the longest extent of the at least one first groove 15.1.

Particularly preferably, the conical spiral has a pitch height of 0.5 mm to 2 mm, preferably 1 mm.

Alternatively or additionally, the spiral-shaped first groove 15.1 has from 1 turn to 3 turns, preferably 1.75 turns. Alternatively or additionally, the spiral-shaped first groove 15.1 has a taper of 0° to 45°, preferably 20°.

FIG. 3 shows a schematic representation of a third embodiment of the cap 1 with the engaging element 9.

The first groove 15.1 can be clearly recognised. In the embodiment of the engaging element shown in FIG. 3, the groove longitudinal extension 17 and the axial direction 19 are aligned parallel to each other, in particular they preferably coincide.

Preferably, the engaging element 9 additionally comprises—in particular on the engaging protrusion 10—at least one second groove 15.2, which extends in a plane, wherein a normal direction of the plane is aligned along the groove longitudinal extension 17 of the first groove 15.1. Particularly preferably, the normal direction of the plane and the groove longitudinal extension 17 are aligned parallel to each other.

In addition, the cap 1—in particular the engaging element 9—has a sealing lip 31 extending in the circumferential direction. The sealing lip 31 is configured on the engaging element 9 and arranged in such a way that the sealing lip 31 comes to rest on an end face 33 of the distal opening 13 of the hollow medical body 3 when the cap 1 is arranged on the hollow medical body 3 in the closing position.

FIG. 4 shows a schematic representation of a fourth embodiment of the cap 1 with the engaging element 9.

The first groove 15.1, in which the groove longitudinal extension 17 and the axial direction 19 in particular form a positive angle, the second groove 15.2 and the sealing lip 31 extending in the circumferential direction can be clearly recognised.

In particular, the normal direction of the plane in which the second groove 15.2 extends includes an angle of 0° to 18° when the cap 1 is arranged on the medical hollow body 3 in the closing position and the axial direction 19 of the medical hollow body 3.

FIG. 5 shows a schematic representation of a first embodiment of a hollow medical body 3 with the cap 1 arranged on the hollow medical body 3 in the closing position.

The engaging element 9 is configured and arranged in such a way that it engages at least partially—in particular with the particularly conically configured engaging protrusion 10—in the distal opening 13 of the hollow medical body 3 when the cap 1 is arranged on the hollow medical body 3 in the closing position. In addition, the groove longitudinal extension 17 is aligned along the axial direction 19 of the medical hollow body 3 when the cap 1 is arranged on the medical hollow body 3 in the closing position.

In particular, the cap 1 has a sealing element 7 in addition to the engaging element 9. The sealing element 7 is configured in such a way that it is in sealing contact with at least a distal area 11 of the medical hollow body 3 when the cap 1 is arranged on the medical hollow body 3 in the closing position.

In the embodiment shown in FIG. 5, the sealing element 7 and the engaging element 9 are configured in two-pieces.

Preferably, the sealing element 7 comprises at least one thermoplastic elastomer (TPE). Alternatively, the sealing element 7 is particularly preferably made of at least one thermoplastic elastomer (TPE).

Preferably, the engaging element 9 comprises at least one elastomer, in particular at least one rubber. Alternatively, the engaging element 9 is particularly preferably made of at least one elastomer, in particular at least one rubber.

Particularly preferably, the sealing element 7 and the engaging element 9 are configured and arranged in such a way that they are spaced apart from one another in at least one first area 39.1 in the direction of the groove longitudinal extension 17. In addition, the sealing element 7 and the engaging element 9 preferably come to rest on one another in at least one second area 39.2—in a point-like or flat manner. The second area 39.2 serves in particular as a spring element that presses the engaging element 9 against the end face 33 of the medical hollow body 3 in the closing position.

FIG. 6 shows a schematic representation of a fifth embodiment of the cap 1 for a medical hollow body 3. The cap 1 comprises a base body 5, the sealing element 7 and the engaging element 9. The sealing element 7 is arranged in the base body 5 in such a way that it is in sealing contact with at least the distal area 11 of the hollow medical body 3 when the cap 1 is arranged on the hollow medical body 3 in a closing position. In addition, the engaging element 9 is configured and arranged in the base body 5 in such a way that it engages at least partially—in particular with the engaging protrusion 10, which is configured conically in particular—in the distal opening 13 of the hollow medical body 3 when the cap 1 is arranged on the hollow medical body 3. Furthermore, the engaging element 9—in particular the particularly conically configured engaging protrusion 10—comprises at least the first groove 15.1 with the groove longitudinal extension 17. When the cap 1 is arranged on the medical hollow body 3 in the closing position, the groove longitudinal extension 17 is aligned along the axial direction 19 of the medical hollow body 3; in particular, when the cap 1 is arranged on the medical hollow body 3 in the closing position, the groove longitudinal extension 17 and the axial direction 19 of the medical hollow body 3 are aligned parallel to each other.

Preferably, the base body 5 at least partially surrounds the sealing element 7 and the engaging element 9.

Particularly preferably, the base body has an upper part 21.1 and a lower part 21.2. In addition, the upper part 21.1 and the lower part 21.2 are preferably connected to each other by means of a plurality of ribs 23.

Preferably, the engaging element 9—in particular the especially conically configured engaging protrusion 10—has at least one second groove 15.2, which extends in a plane, wherein a normal direction of the plane is aligned along the groove longitudinal extension. Particularly preferably, the normal direction of the plane and the groove longitudinal extension are aligned parallel to each other.

In the embodiment shown here, the sealing element 7 and the engaging element 9 are configured single-piece, in particular with the same material.

Furthermore, the base body 5, in particular the lower part 21.2 of the base body 5, preferably has an internal thread 25 which engages around the distal area 11 of the medical hollow body 3 when the cap 1 is arranged on the medical hollow body 3 in the closing position. In addition, the internal thread 25 is particularly preferably configured as a Luer thread.

Preferably, the internal thread 25, in particular the Luer thread, and the base body 5, in particular the lower part 21.2, are configured in two-pieces. Alternatively, the internal thread 25, in particular the Luer thread, and the base body 5, in particular the lower part 21.2, are preferably configured as a single-piece.

Furthermore, the base body 5, in particular the upper part 21.1 of the base body 5, preferably comprises at least one recess 27. In addition, the sealing element 7 preferably comprises at least one anchoring element 29, wherein the sealing element 7 is arranged in the base body 5 in such a way that the at least one anchoring element 29 engages in the at least one recess 27.

Preferably, the sealing element 7 comprises at least one thermoplastic elastomer (TPE). Alternatively, the sealing element 7 particularly preferably consists of at least one thermoplastic elastomer (TPE).

FIG. 7 shows a schematic representation of a sixth embodiment of the cap 1 for the medical hollow body 3.

In addition to the fifth embodiment of the cap 1 shown in FIG. 6, the sixth embodiment of the cap 1 comprises the sealing lip 31 extending in the circumferential direction. The sealing lip 31 is configured on the engaging element 9 and arranged in such a way that the sealing lip 31 comes to rest on the end face 33 of the distal opening 13 of the hollow medical body 3 when the cap 1 is arranged on the hollow medical body 3 in the closing position.

FIG. 8 shows a schematic representation of a second embodiment of the hollow medical body 3 with the cap 1 arranged on the hollow medical body 3 in the closing position.

The cap 1 preferably has a separate retaining element 35. The retaining element 35 is preferably arranged on the base body 5 of the cap 1 in such a way that it grips the medical hollow body 3 in a form and/or force-fit manner when the cap 1 is arranged on the medical hollow body 3 in the closing position.

Preferably, the base body 5, in particular the upper part 21.1 of the base body 5, has a plurality of recesses 27, in particular seven recesses 27. In addition, the sealing element 7 has a plurality of anchoring elements 29, in particular seven anchoring elements 29, in particular the number of recesses 27 and the number of anchoring elements 29 are identical. Preferably, at least one anchoring element 29 of the plurality of anchoring elements 29 is configured as a radial anchoring element 29.1, in particular the sealing element 7 has six radial anchoring elements 29.1, which protrudes from the respective recess 27 in a radial direction. In addition, preferably at least one anchoring element 29 of the plurality of anchoring elements 29 is configured as an axial anchoring element 29.2, in particular the sealing element 7 has an axial anchoring element 29.2 which projects in an axial direction out of the associated recess 27. For a clearer representation, only a radial anchoring element 29.1 and the axial anchoring element 29.2 with the respective associated recesses 27 are provided with reference signs.

Furthermore, the base body 5, in particular the lower part 21.2 of the base body 5, preferably comprises a first gripping mould 37.1 and a second gripping mould 37.2, wherein the first gripping mould 37.1 and the second gripping mould 37.2 are configured on a lateral surface of the base body 5. Particularly preferably, the first gripping mould 37.1 and the second gripping mould 37.2 are arranged diametrically opposite one another.

FIG. 9 shows a schematic representation of a third embodiment of the medical hollow body 3 with the cap 1, which is arranged on the medical hollow body 3 in the closing position. FIG. 9 a) shows a first view and FIG. 9 b) shows a second view of the medical hollow body 3, wherein the second view emerges from the first view by a rotation of the medical hollow body 3 about the axial direction 19 by preferably 90° when the first groove 15.1 is rotated backwards into the image plane.

The sealing element 7 and the engaging element 9 are preferably configured in two pieces, wherein the engaging element 9 particularly preferably comprises the first groove 15.1, the second groove 15.2 and the sealing lip 31.

In FIG. 9 a), one of the recesses 27 can be seen on an end face of the base body 5, wherein the axial anchoring element 29.2, which is configured in particular on the sealing element 7, protrudes from the recess 27 in the axial direction 19.

Furthermore, the radial anchoring elements 29.1 are not visible in FIG. 9 a).

In FIG. 9 b), analogous to FIG. 8, six recesses 27 can be seen on the lateral surface of the base body 5, in particular on the lateral surface of the upper part 21.1 of the base body 5, wherein a radial anchoring element 29 engages in each recess 27 of the six recesses 27 and protrudes radially. For a clearer representation, only one radial anchoring element 29.1 and the associated recess 27 are provided with reference signs.

The first groove 15.1 of the engaging element 9 is not visible in FIG. 9 b) due to the rotated view.

Furthermore, the first gripping mould 37.1 and the second gripping mould 37.2 are not visible in FIG. 9 b).

A comparison of the two views from FIG. 9 a) and FIG. 9 b) shows that both the radial anchoring elements 29.1 and the first gripping mould 37.1 and the second gripping mould 37.2 are only configured in partial areas in the circumferential direction.

FIG. 10 shows a schematic representation of a third embodiment of the medical hollow body 3 with the cap 1.

FIG. 10 shows an external view of the hollow medical body 3 with the cap 1. It is therefore possible that the sealing element 7 and the engaging element 9 are configured single-piece with one another. Alternatively, however, it is also possible that the sealing element 7 and the engaging element 9 are configured in two-pieces.

The axial anchoring element 29.2 is preferably configured as an arrow in order to advantageously indicate a particularly preferred opening direction.

Preferably, the radial anchoring elements 29.1 are configured only in partial areas along the circumferential direction of the lateral surface of the upper part 21.1 of the base body 5. Alternatively or additionally, the first gripping mould 37.1 is configured only in partial areas along the circumferential direction of the lateral surface of the lower part 21.2 of the base body 5. Preferably, the radial anchoring elements 29.1, the first gripping mould 37.1 and the second gripping mould 37.2 are configured in such a way that the cap 1 can be gripped easily and securely.

FIG. 11 shows a schematic sectional view of a seventh embodiment of the cap 1 with the retaining element 35, which is arranged in particular on a lower part 21.2 of the base body 5.

Preferably, the retaining element 35 comprises an anti-rotation section 41 which engages in a notch 43 of the base body 5, in particular of the lower part 21.2 of the base body 5. In particular, the retaining element 35 is pushed into the base body 5, in particular into the lower part 21.2 of the base body 5, during assembly of the cap 1—pushed in from the left in the figure shown.

Preferably, the retaining element 35 comprises a clamping geometry, in particular a plurality of teeth 45, which is configured and arranged in such a way that the medical hollow body 3, in particular the distal area 11 of the medical hollow body 3, can be gripped in a form and/or force-fit manner. An inner diameter 47 of the retaining element 35 is particularly preferably smaller than a diameter of the distal area 11 of the hollow medical body 3 on which the cap 1 is arranged. Due to the plurality of teeth 45, the retaining element 35 is flexible in partial areas, so that the cap 1 can be arranged on the medical hollow body 3 in the closing position.

Particularly preferably, the retaining element 35 is latched to the base body 5 before the cap 1 is arranged in the closing position on the medical hollow body 3.

FIG. 12 shows a flow diagram of a first embodiment of a method for manufacturing the cap 1.

In a first initial step a1), the base body 5 and the sealing element 7 are produced by means of a two-component injection moulding process. Preferably, the sealing element 7 is manufactured from at least one thermoplastic elastomer.

In a second first step a2), the engaging element 9 is preferably produced from at least one elastomer. Preferably, the at least one elastomer is a vulcanisate of a rubber and/or a vulcanisate of a silicone rubber.

In a second step b), the engaging element 9 is arranged in the base body 5 and the sealing element 7, wherein the cap 1 is produced.

In an optional third step c), the retaining element 35 is preferably arranged on the base body 5. Particularly preferably, the retaining element 35 is connected to the base body 5 by the at least one anti-rotation section 41 engaging in the at least one notch 43 of the base body 5.

In an optional fourth step d), the cap 1 is preferably arranged in the closing position on the medical hollow body 3.

FIG. 13 shows a flow diagram of a second embodiment of a method for manufacturing the cap 1.

In a first step a), the base body 5, the sealing element 7 and the engaging element 9 are produced by means of a two-component injection moulding process or a multi-component injection moulding process, wherein the sealing element 7 and the engaging element 9 are configured in a single-piece with one another. Preferably, the single-piece configuration of the sealing element 7 and the engaging element 9 is produced from at least one thermoplastic elastomer.

Thus, the cap 1 is produced in a single step, in particular the first step a).

In the optional third step c), the retaining element 35 is preferably arranged on the base body 5. Particularly preferably, the retaining element 35 is connected to the base body 5 by the at least one anti-rotation section 41 engaging in at least one notch 43 of the base body 5.

In the optional fourth step d), the cap 1 is preferably arranged in the closing position on the medical hollow body 3.

1. Cap for a medical hollow body, with
an engaging element which is configured such that it engages at least partially in a distal opening of the hollow medical body when the cap is arranged on the hollow medical body in the closing position, wherein
the engaging element has at least one first groove, wherein a groove direction associated with the at least one first groove is aligned along an axial direction of the medical hollow body when the cap is arranged on the medical hollow body in the closing position.

2. Cap according to claim 1, wherein the at least one first groove extends straight and has a groove longitudinal extension as the groove direction, wherein preferably the groove longitudinal extension encloses an angle of 0° to 25° when the cap is arranged on the hollow medical body in the closing position and the axial direction of the hollow medical body.

3. Cap according to claim 1, wherein the at least one first groove is configured along a spatial curve, in particular a helical line or a conical spiral, wherein the spatial curve extends around a winding axis, preferably the winding axis of the at least one first groove and the axial direction of the hollow medical body are parallel to each other when the cap is arranged on the hollow medical body in the closing position.

4. Cap according to claim 2, wherein the engaging element has at least one second groove which extends in a plane, wherein a normal direction of the plane when the cap is arranged on the hollow medical body in the closing position and the axial direction of the hollow medical body enclose an angle of 0° to 18°.

5. Cap according to claim 1, having a sealing element, wherein the sealing element is configured in such a way that it bears in a sealing manner against at least one distal area of the hollow medical body when the cap is arranged on the hollow medical body in a closing position.

6. Cap according to claim 1, wherein the sealing element and the engaging element are configured single-piece with each other.

7. Cap according to claim 1, wherein the sealing element and the engaging element are configured in two-pieces.

8. Cap according to claim 1, wherein the engaging element has a sealing lip which extends in the circumferential direction and is arranged on the engaging element in such a way that the sealing lip comes to rest on an end face of the distal opening of the hollow medical body when the cap is arranged on the hollow medical body in the closing position.

9. Cap according to claim 1, with a base body, wherein the engaging element and/or the sealing element are arranged at least partially within the base body.

10. Cap according to claim 1, wherein the base body has an internal thread which engages around a distal area of the medical hollow body when the cap is arranged on the medical hollow body in the closing position, wherein the thread is preferably a Luer thread.

11. Cap according to claim 1, wherein the sealing element and the engaging element are arranged in the base body such that they are spaced apart from each other in at least one first area along the groove longitudinal extension, wherein they come to rest on each other in at least one second area.

12. Cap according to claim 1, wherein
the base body comprises an upper part and a lower part, wherein
the upper part and the lower part are connected by means of a plurality of ribs, wherein preferably
the lower part comprising the internal thread.

13. Cap according to claim 1, having a retaining element which is arranged on the base body in such a way that it preferably engages around the medical hollow body in a form and/or force-fit manner when the cap is arranged on the medical hollow body in the closing position, wherein the retaining element has at least one anti-rotation section which engages in a recess of the base body.

14. Cap according to claim 1, wherein the base body and the sealing element and preferably the engaging element are produced by means of a two-component injection moulding process.

15. Cap according to claim 1, wherein the sealing element comprises at least one thermoplastic elastomer, preferably consists of at least one thermoplastic elastomer.

16. Cap according to claim 1, wherein the engaging element comprises at least one elastomer, preferably consists of at least one elastomer.

17. Cap according to claim 1, wherein
the base body comprising a first gripping mould and a second gripping mould, wherein
the first gripping mould and the second gripping mould are configured on a lateral surface of the base body, wherein preferably,
the first gripping mould and the second gripping mould are arranged diametrically opposite one another, wherein preferably
the first gripping mould and the second gripping mould are configured on the lower part of the base body.

18. Cap according to claim 1, wherein
the base body comprises at least one recess, wherein
the sealing element comprises at least one anchoring element, wherein
the sealing element is arranged in the base body in such a way that the at least one anchoring element engages in the at least one recess and preferably projects out of the at least one recess in a preferably axial and/or radial direction.

19. Medical hollow body with a cap according to claim 1.

20. Method for producing a cap according to claim 1, wherein the base body and the sealing element and preferably the engaging element are produced by means of a two-component injection moulding process.
